(19) United States
(12) Patent Application Publication
(10) Pub. No.: US 2021/0244637 A1
(43) Pub. Date: Aug. 12, 2021

(54) DEUTERATED POLYUNSATURATED FATTY ACIDS OR ESTERS THEREOF FOR COSMETIC APPLICATIONS

(71) Applicant: Retrotope, Inc., Los Altos, CA (US)

(72) Inventor: Mikhail Sergeevich SHCHEPINOV, Oxford (GB)

(21) Appl. No.: 17/174,054

(22) Filed: Feb. 11, 2021

Related U.S. Application Data
(60) Provisional application No. 62/975,543, filed on Feb. 12, 2020.

(51) Int. Cl. A61K 8/36; A61K 31/201; A61K 31/202; A61Q 19/08; A61K 45/06; A61K 9/00

(52) U.S. Cl. CPC A61K 8/361; A61K 31/201; A61K 9/0014; A61Q 19/08; A61K 45/06; A61K 31/202

(57) ABSTRACT

Disclosed are cosmetic compositions and skin care products that are stabilized against lipid chain auto-oxidation. In particular, this invention substitutes a portion of polyunsaturated fatty acids used in cosmetic compositions and skin care products with a corresponding deuterated polyunsaturated fatty acid.

### CROSS-REFERENCED TO RELATED APPLICATIONS

This application claims priority to U.S. Provisional application Ser. No. 62/975,543, filed Feb. 12, 2020, under the provisions of 35 U.S.C. § 119 and is incorporated herein by reference in its entirety.

### BACKGROUND OF THE INVENTION

### Field of the Invention

This invention is directed to cosmetic compositions and skin care products that are stabilized against lipid auto-oxidation. In particular, this invention substitutes a portion of polyunsaturated fatty acids used in cosmetic compositions with a corresponding deuterated polyunsaturated fatty acid.

### State of the Art

Aging is an accumulation of physical changes over time that result from cumulative damage to tissues that overwhelm the body's natural ability to repair them. According to free radical theory, such damage is caused by reactive oxygen species (ROS) that are a side product of oxidative phosphorylation, a key process for energy production by cells. ROS oxidatively damage cellular components, gradually leading to functional decline.

Environmental (extrinsic) factors such as exposure to direct sunlight or smoking, among others, may accelerate some aspects of the aging process such as skin aging. To address this, cosmetic skin compositions are formulated as anti-aging creams and moisturizers. These products are promoted to retain the youthful nature of the skin and/or to rejuvenate aged skin look younger and to reduce the number of wrinkles.

The science of skin aging is a progressive, physiological process resulting in loss in homeostatic capacity. Skin wrinkles are the result of structural changes in the dermis and the subcutaneous tissue which leads to the formation of wrinkles. Histologically, there are four main types of wrinkles, each developing o the specific skin regions: atrophic crinkling rhytids, permanent elastotic creases, dynamic expression lines, and gravitational folds. Each type of wrinkle responds differently to cosmetic and dermatological treatments.

Highly visual age associated skin changes are perceived to be a serious beauty problem. The conspicuous nature of the problem is caused by several tell-tale signs of skin aging that include discoloration, texture/elasticity loss and wrinkles. These, and to some extent a related phenomenon of cellulite formation results from structural and metabolic changes in changes in the connective tissue that are accelerated by ROS damage. Such aged skin appears markedly changed at a histological level. Oxidative stress also influences other parameters related to skin health, such as sebum content, pH, and hydration. Lips ae a particular example of the latter.

Exposure to the sun's rays leads to accelerated skin aging and photo aging. Two major UV components of the sun light are UVA (400-320 nm, glass transparent is in this region) and UVB (320-280 nm; filtered out by glass). Sunlight reaching the skin contains 10-100 times more UVA than UVB depending on environmental factors. UVB is mostly responsible for sunburns (SPF sunscreen rating system is calibrated for UVB. UVB causes many skin cancers, but melanoma (formed b melanocytes) is caused by UVA which penetrates deeper into the skin, reach the base of the epidermis that contains melanocytes. At high latitudes UVA levels do not decrease by as much as UVB levels. Melanin protects against UV but is oxidised in the process, forming melanin radicals which then generate other radicals. Red-haired people are more likely to develop melanomas since they only produce a red-yellow pigment pheomelanin and no black-brown pigment eumelanin. Pheomelanin is less stable of the two and is more likely to generate radicals. The photo aging process is also initiated by the oxidative damage, inflicted by UV-generated ROS. Sun screens reduce this damage by blocking UV absorption and/or neutralizing ROS.

A characteristic odor is another feature associated with the aged skin. Many animals rely on smell to discern between young and old individuals. There is a substantial difference in the skin smell between younger and older subjects. It has been reported in a Japanese study that people over 40 have an unpleasant “aging odor” which was attributed mainly to products of oxidation of unsaturated fatty acids such as nonenal. A more recent study found lower amounts of nonenal (which has a “greasy” smell) and lipid peroxides in aged Americans. This was attributed to the dietary differences between Japanese and American diets, mainly the much larger intake of oxidation-prone essential fatty acids by the Japanese population from a seafood-rich diet.

Poly-unsaturated fatty acids (PUFAs) play a major role in age associated skin changes. Fatty acids, which are the main component of lipid membranes, bear the first brunt of ROS attack. The ensuing peroxidation gives rise to reactive intermediates that carry out further damage and change the properties of lipid membranes. Peroxidized PUFAs initiate further oxidation chain reactions, leading to oxidative damage of proteins. Even oxidative DNA damage and mutagenesis (aging of the genome) have been shown to be induced by lipid peroxidation. Moreover, some products of PUFAs oxidation, such as nonenal (VS) and malondialdehyde, form cross-links in proteins and other cellular components (advanced glycation products (AGES), leading to chemical debris that accumulates with age (such as the age pigment lipofuscin) and further complicating the repair processes. Lipid peroxidation may also be involved in cancer related neoplastic transformations. On a macro-level, linoleic, and to a smaller extent, linolenic acid deficiency causes dry skin, dermatitis, and massive transepidermal water loss.

Based on the above, topical cosmetic compositions that inhibit or prevent lipid auto-oxidation in the dermal layer would provide meaningful inhibition of wrinkle formation and other characteristics of aged skin.

### SUMMARY OF THE INVENTION

This invention is directed to topical cosmetic and skin care compositions formulated to inhibit lipid peroxidation thereby reducing or preventing such peroxidation as a driving component in aging skin and causing wrinkles. This invention is based, in part, on the recognition that oxidative damage, whether triggered by intrinsic (metabolically-generated) or extrinsic (environmentally-generated) ROS, the oxidative damage nearly always occurs at the same specific molecular sites—namely at the bis-allylic methylene groups in the fatty acids. This damage initiates further detrimental cascades of oxidation. Further, the most important PUFAs (linoleic and linolenic acids) belong to the group of essential nutrients, i.e., they cannot be biosynthesized by humans and have to be provided through the diet, skin adsorption, or other exogenous administration.

The deuterated PUFAs contained in these topical compositions integrate into the dermal cells including the cell membrane comprises thereby stabilizing these cells against lipid chain auto-oxidation. Such, in turn, reduces damage to the skin arising from such auto-oxidation thereby reducing the adverse cosmetic changes to skin discussed above. Unlike prior disclosures which required ingestion of deuterated PUFAs, this invention relies upon direct absorption of deuterated PUFAs into the skin after topical application in sufficient amounts to be beneficial.

Based on the above, in one embodiment, this invention provides for a cosmetic or skin care composition comprising an effective amount of one or more deuterated PUFAs said deuterated PUFA being sufficient to inhibit or prevent lipid auto-oxidation and subsequent damage to the dermis of the skin. Preferably, the deuterated PUFA employed in said compositions is a deuterated linoleic acid or an ester thereof or a deuterated linolenic acid or an ester thereof.

Fatty acids and mixtures of fatty acids such as stearic acid, oleic acid, lauric acid, palmitic acid, and/or myristic acid are used in cosmetic and skin care products such as creams, lotions, ointments, emulsions, cakes, soaps, pastes, and the like. These include cosmetics, face creams, sunscreens, body creams, anti-aging creams, and the like. Deuterated polyunsaturated fatty acids or esters can be used in any cosmetic composition or skin care products that contain fatty acids, fatty acid esters, or natural oils merely by replacing a portion of the fatty acids, fatty acid esters or natural oils in these compositions/products with the deuterated polyunsaturated fatty acids or esters. In one embodiment, the amount of fatty acids, fatty acid esters, or natural oils replaced by one or more deuterated polyunsaturated fatty acids or esters ranges from about 0.1 to about 30 weight percent based on the total weight of the fatty acids, fatty acid esters, and natural oils present in the composition.

In another embodiment, the amount of deuterated polyunsaturated fatty acid or esters thereof ranges from about 0.1 to about 25 weight percent based on the total weight of the fatty acids, fatty acid esters, and natural oils present in the composition. In another embodiment, the amount of deuterated polyunsaturated fatty acid or esters thereof range from about 0.1 to about 20 weight percent based on the total weight of the fatty acids, fatty acid esters, and natural oils present in the composition.

In yet another embodiment, this invention provides for a method for reducing dermal degradation of a patient's skin which method comprises applying to the skin a cosmetic or skin care composition comprising an effective amount of one or more deuterated PUFAs in an amount sufficient to inhibit or prevent lipid auto-oxidation and subsequent damage to the dermis of the skin; and allowing adsorption of at least a portion of said deuterated PUFAs into the skin. Such methods can be used to attenuate the smell of aged skin.

In one preferred embodiment, the deuterated fatty acid or ester is 11,11-D2-linoleic acid or ester; or is 8,8,11,11-D4-linoleic acid or ester; or is 11,11-D2-linolenic acid or ester; or is 11,11,14,14-linolenic acid or ester optionally having additional deuteration elsewhere in the molecule.

### DETAILED DESCRIPTION OF THE INVENTION

This invention provides for cosmetic compositions and skin care products that are stabilized against lipid auto-oxidation. Specifically, this invention provides for a portion of deuterated polyunsaturated fatty acids or esters in these compositions products to stabilize the dermis against lipid auto-oxidation.

However, prior to describing this invention in further detail, the following terms will first be defined.

The term “bis-allylic position” refer to the hydrogen and carbon atoms positioned between between two vinyl groups (i.e., —CH═CH—CH₂—CH═CH2-).

The term “lipid auto-oxidation” refers to the well-known process regarding polyunsaturated fatty acid peroxidation. Such occurs when once a first polyunsaturated fatty acid is oxidized by a ROS, a cascade of further oxidation of other polyunsaturated fatty acid groups in the lipid membrane occurs. This is because a single ROS generates oxidation of a first polyunsaturated fatty acid through a free radical mechanism which, in turn, can oxidize a neighboring polyunsaturated fatty acid in a phospholipid through the same free radical mechanism which yet again can oxidize another neighboring polyunsaturated acid in a process referred to as lipid chain auto-oxidation. The resulting damage includes a significant number of oxidized polyunsaturated fatty acids in, e.g., phospholipid components found in the cell membrane.

As used herein, the term “linoleic acid” refers to the compound and a pharmaceutically acceptable salt thereof having the formula provided below and having the natural abundance of deuterium at each hydrogen atom:

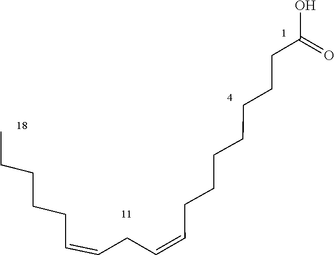 [SMILES: CCCCC/C=C\C/C=C\CCCCCCCC(=O)O]

Linoleic acid has a single bis-allylic methylene group at carbon 11. Esters of linoleic acid are formed by replacing the —OH group with —OR. Such esters are as defined herein below.

As used herein, the term “linolenic acid” refers to the compound and a pharmaceutically acceptable salt thereof having the formula provided below and having the natural abundance of deuterium at each hydrogen atom:

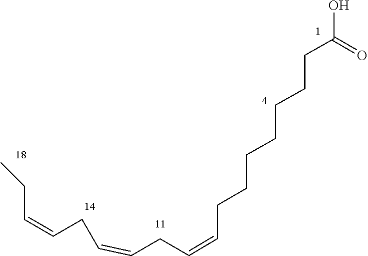 [SMILES: CC/C=C\C/C=C\C/C=C\CCCCCCCC(=O)O]

Linolenic acid has two bis-allylic methylene groups at carbon 11 and 14. Esters of linolenic acid are formed by replacing the —OH group with —OR. Such esters are as defined herein below.

As used herein, arachidonic acid has the numbering system as described below:

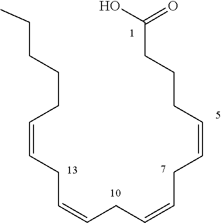 [SMILES: CCCCC/C=C\C/C=C\C/C=C\C/C=C\CCCC(=O)O]

Arachidonic acid has three bis-allylic methylene groups at carbon 7, 10 and 13. Esters of linolenic acid are formed by replacing the —OH group with —OR. Such esters are as defined herein below.

The term “deuterated polyunsaturated fatty acids” refers to those well-known PUFAs having at least one hydrogen atom at the methylene group found at a bis-allylic position replaced by a deuterium atom and optionally non-exchangeable deuterium atoms at other positions within the molecule. Still further, such deuterated polyunsaturated fatty acids can have ¹²C replaced at the bis-allylic position replaced with ¹³C. Suitable deuterated PUFAs include deuterated linoleic acid and esters thereof; deuterated linolenic acid and esters thereof; as well as higher order deuterated PUFAs such as deuterated arachidonic acid.

As used herein and unless the context dictates otherwise, the term “deuterated linoleic acid or an ester thereof” refers to linoleic acid or ester compounds having at least one deuterium atom at the methylene group found at the bis-allylic position and optionally additional non-exchangeable deuterium atoms at other positions within the molecule.

Specific compounds encompassed by this definition include, by way of example only, 11-D1-linolenic acid, 11,11-D2-linolenic acid, 11,14-D2-linolenic acid, 14-D1-linolenic acid, 14,14-D2-linolenic acid, 11,11,14-D3-linolenic acid, 11,14,14-D3-linolenic acid, 11,11,14,14-D4-linolenic acid, and perdeuterated linoleic acid.

As used herein and unless the context dictates otherwise, the term “deuterated linolenic acid or an ester thereof” refers to linolenic acid or ester compounds having at least one deuterium atom at a methylene group found at one of the two bis-allylic positions and optionally additional non-exchangeable deuterium atoms at other positions within the molecule. Specific compounds encompassed by this definition include, by way of example only, 11-D1-linoleic acid, 11,11-D2-linoleic acid, 8,11-D2-linoleic acid, 8,11,11-D3-linoleic acid, 8,8,11-D3-linoleic acid, 8,8,11,11-D4-linoleic acid, and perdeuterated linoleic acid.

As used herein and unless the context dictates otherwise, the term “deuterated arachidonic acid or an ester thereof” refers to arachidonic acid or ester compounds having at least one deuterium atom at a bis-allylic position and optionally additional non-exchangeable deuterium atoms at other positions within the molecule. Specific compounds encompassed by this definition include, by way of example only, 7,7-D2-arachidonic acid, 10,10-D2-arachidonic acid, acid, 13,13-D2-arachidonic acid, 7,7,10,10-D4-arachidonic acid, 7,7,13,13-D4-arachidonic acid, 10,10,13,13-D4-arachidonic acid, 7,7,10,10,13,13-D6-arachidonic acid and perdeurerated arachidonic acid.

As used herein, the term “ester” means any pharmaceutically acceptable ester of a deuterated PUFA such as but not limited to C₁-C₆ alkyl esters, glycerol (including monoglycerides, diglycerides and triglycerides), sucrose esters, phosphate esters, and the like. The particular ester employed is not critical provided that the ester is pharmaceutically acceptable (non-toxic and biocompatible).

As used herein, the term “phospholipid” refers to any and all phospholipids that are components of a dermal cell. Included within this term are phosphatidylcholine, phosphatidylethanolamine, phosphatidylserine, and sphingomyelin. Dermal cells are particularly enriched in phospholipids comprising linoleic acid.

The term “non-exchangeable” refers to deuterium atom attachment to the carbon atom where exchange from one molecule to another molecule can occur by hydrogen bonding interactions. An example of an exchangeable hydrogen/deuterium atom is shown below.

CH₃CH₂—COOH+D₂O [symbol] CH₃CH₂—COOD+DHO

Here, the carboxylic acid proton of propionic acid is exchangeable with D₂O as it is subject to hydrogen bonding interchange whereas the methylene and methyl hydrogens do not.

The term “natural oils” refers to those well-known oils such as coconut oil, olive oil, sunflower seed oil, as well as essential oils such as lemon oil, lavender oil, oil of cinnamon, and the like. These oils are well known for their use in cosmetic compositions and skin care products.

The term “skin care products” means any topically applied product that is applied to the skin for principally for dermatological (medicinal) purposes whether in the form of a cream, a lotion (including emulsions), ointments, pastes, thixotropic compositions, soaps, emollients, and the like. Such dermatological purposes include, but not limited to, the treatment of rashes, dry skin, scratches, minor cuts, bruises, acne, facial hair, dermatitis, hair loss, and the like.

The term “cosmetic” means any topically applied product that is applied to the skin principally for cosmetic purposes whether in the form of a cream, a lotion (including emulsions), ointments, pastes, sprays, thixotropic compositions, emollients, and the like. Such cosmetic purposes include, but not limited to, cosmetic application to hide aged spots, to treat or prevent wrinkles, to use as a makeup, to hide bruising or scratches, to moisturize skin, to use as a facial cream or mask, and the like.

As used herein, the term “patient” refers to a human who seeks to topically apply a cosmetic or skin care composition.

As used herein, the term “effective amount” refers to the amount of a composition of this invention that is sufficient to inhibit or prevent lipid auto-oxidation arising from ROS in the dermal layer of a patient.

It is understood that in some cases, the composition could be used as a skin care product and/or as a cosmetic. However, for the purposes of this application, the principle use of the product dictates whether it is a cosmetic or a skin care product.

### Compound Preparation

Deuterated PUFAs are disclosed in a number of references and/or are commercially available. For example, 11-D1-linoleic acid, 11,11-D2-linoleic acid, 8,8,11,11-D4 linoleic acid, 11,11-D2-linolenic acid, 11,11,14,14-D4-linolenic acid and other deuterated PUFAs are known in the art. See, for example, U.S. Pat. Nos. 10,052,299 and 10,730,821 both of which are incorporated herein by reference in their entirety. In addition, 11-D1-linoleic acid is commercially available from Cayman Chemical Company, Ann Arbor, Mich., USA 48108. Still further, 7,7-D2-arachidonic acid, 10,10-D2-arachidonic acid, 13,13-D2-arachidonic acid, 7,7,10,10-D4-arachidonic acid, 7,7,13,13-D4 arachidonic acid, 10,10,13,13-D4-arachidonic acid, 7,7,10,10,13,13-D6-arachidonic acid are disclosed by Shchepinov, et al., Molecules, 28(12):3331 et seq. (2018). Other deuterated arachidonic acid compounds are known in the art.

Esters of these deuterated fatty acids are prepared by conventional techniques well known in the art.

### Compositions

The compositions described herein include any one of those suitable for topical application to the skin and are described below:

Creams are spreadable, topically applied compositions comprising about equal parts of water and oil. The latter includes fatty acids and the like. Creams are conventional and are well known in the art. In fact, certain high-end skin care products employ PUFAs which are described as providing enhancements to the product as compared to saturated fats. Lotions are a subclass of creams and are distinguished as being less viscous due to the addition of more water and, hence, somewhat easier to apply to the skin.

Ointments are also spreadable, topically applied compositions but differ from creams in that they comprise about 4 times as much oil as water. Ointments are also well known in the art. Pastes are another form of ointments but contain a large amount of finely divided solids such as starch, zinc oxide, and calcium carbonate.

Emollients are compositions that are designed to moisturize the skin and are applied directed to the skin to soothe and hydrate it.

Sprays are well known in the art and preferably are film-forming sprays that provide a discernible film after evaporation of the solvent.

In general, the compositions described herein comprise an oily composition comprising:

a) oils containing about 1% to about 99% deuterated PUFAs in appropriate ratios as the primary ROS-protected essential lipid component;
b) at least one hydrating agent; and
c) the balance deionized water.

Preferably, the deuterated PUFA is a deuterated linoleic acid or an ester thereof. The particular amount of deuterated PUFA employed is sufficient to stabilize cell membranes in the dermis against degradation by ROS. In general, the overall composition comprises at least about 0.5% of a deuterated PUFA and, preferably, about 1% based on the total weight of the composition.

In addition, optional components include one or more common additives such as aromas, regulators of lipid metabolism, antioxidants, and the like. Suitable hydrating agents include vegetable glycerin, aloe-vera, and vegetable oils other than grapeseed oil, for example, vitamin E oil, jojoba oil, flaxseed oil, primrose oil or any other botanical essential oil. Each are used in their conventional amounts.

In some embodiments, composition disclosed herein can include one or more cosmetic ingredients. The CTFA International Cosmetic Ingredient Dictionary and Handbook (2004 and 2008) describes a wide variety of non-limiting cosmetic ingredients that can be used in the compositions disclosed herein. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided in the following subsections.

In some embodiments, compositions disclosed herein can include one or more UV absoption agent. UV absorption agents that can be used in combination with the compositions disclosed herein include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. Non-limiting examples of physical sunblocks include, kaolin, talc, petrolatum and metal oxides (e.g., titanium dioxide and zinc oxide). Compositions of the present invention can have UVA and UVB absorption properties. The compositions can have an sun protection factor (SPF) of 2, 3, 4, 56, 7, 8, 9, 10, 11, 12, 13, 14, 15, 20, 25, 30, 35, 40, 45, 50, 55, 60, 70, 80, 90 or more, or any integer or derivative therein.

In some embodiments, the compositions disclosed herein can include one or more moisturizing agents. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Further examples include acetylated lanolin, acetylated lanolin alcohol, alanine, algae extract, aloe barbadensis, aloe-barbadensis extract, aloe barbadensis gel, althea officinalis extract, apricot (Prunus armeniaca) kernel oil, arginine, arginine aspartate, arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamon (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Xea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (Oenothera biennis) oil, fatty acids, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (Mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

In some embodiments, compositions disclosed herein can include one or more antioxidants. Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In some embodiments, compositions disclosed herein can include one or more structuring agents. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

In some embodiments, compositions disclosed herein can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

In some embodiments, compositions disclosed herein can include one or more silicone containing compounds. In non-limiting aspects, silicone containing compounds can include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids. The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the compositions herein include those available from General Electric Co., Silicone Products Div., Waterford, N.Y. and SWS Silicones Div. of Stauffer Chemical Co., Adrian, Mich.

In some embodiments, the compositions disclosed herein can include one or more essential oils. Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096. Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

In some embodiments, the compositions disclosed herein can include one or more thickening agents. Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene or trihydroxystearin, or a mixture of both. Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich). Non-limiting examples of crosslinked polyacrylate polymers include cationic and nonionic polymers. Examples are described in U.S. Pat. Nos. 5,100,660; 4,849,484; 4,835,206; 4,628,078; 4,599,379). Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids. Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit. Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

In some embodiments, compositions disclosed herein can include on or more preservatives. Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

In some embodiments, the compositions disclosed herein can include one or more further pharmaceutical ingredients/active agents. Non-limiting examples of pharmaceutical ingredients/active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, and the like.

### Methods

The methods of this invention utilize periodic application of a composition described herein to at least a portion of the skin. It is well known that the phospholipids in a cell are replaced over time with a small percentage of turnover on a daily basis. Accordingly, periodic or daily application of the composition allows the deuterated PUFAs to be absorbed into the cells. Over repeated usage, the concentration of deuterated PUFAs in the dermal cells is sufficient to inhibit lipid chain auto-oxidation.

Periodic application preferably includes twice daily, once daily, or several times over a week. Consistent application is required in order to retain an effective amount of deuterated PUFAs in the dermal cells. As noted above, the phospholipids in a cell turnover over time and consistent use ensures that as phospholipids comprising deuterated PUFAs are replaced, there is a source of phospholipids comprising deuterated PUFAs available. Stated another, the failure to practice consistent application will result in either a diminished or complete loss of the beneficial results described herein.

One particular advantage arising from consistent application of these compositions is a reduction in skin odor associated with age and degradation of the fatty acids in the cell attributed at least in part by ROS. As a person ages, the ability to scavenge ROS diminishes and this results in higher rates of lipid chain auto-oxidation to occur. In the skin, the resulting auto-oxidative degradation results in a characteristic smell associated with aged skin. In many individuals, this smell is quite unpleasant. Methods employing compositions of this invention can attenuate that smell by stabilizing phospholipids in the dermal cells from such degradation. These compositions can preferably take the form of a soap, a body wash, a lotion, or a cream.

### Pharmaceutical Compositions

The compositions described herein can be used for medicinal purposes such as treating dry skin, treating scarring, treating aged skin, and to treat sweat gland in the form of a deodorant and the like. Such medicinal purposes include both prescription-based and over-the-counter based products.

The compositions can comprise a further medicament in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art. Acceptable medicaments include anti-itch, anti-scarring and other conventional drugs used in topical compositions. In addition, anti-oxidants such as edaravone, idebenone, mitoquinone, mitoquinol, vitamin C, or vitamin E can be included in appropriate amounts in the compositions described herein.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying FIGURES. Such modifications fall within the scope of the appended claims. In these examples, the following terms are used herein and have the following meanings. If not defined, the abbreviation has its conventional medical meaning.

D2-AA=13,13-D2-Arachidonic Acid

AA=Arachidonic Acid

D2-LA=11,11-D2-Linoleic Acid

LA=Linoleic Acid

### Example 1—Reduction in Skin Odor

Mice are known to generate skin odor and the causative agent is 2-nonenal. This example explores the contribution of lipid chain auto-oxidation to the generation of such skin odor. Specifically, two cohorts of BALB/c mice are tested. The feed of the first cohort is supplemented daily with D2-LA ethyl ester in an amount such that D2-LA ethyl ester constitutes no more than about 10% of the PUFAs ingested. The feed provided to the other cohort is supplemented with LA ethyl ester in otherwise identical amounts. After 4 weeks of dosing, the urine from each cohort is collected over a 2-day period and then analyzed to assess differences in the amount of 2-nonenal. Alternatively, skin samples can be evaluated for sebum exudates such as by swabs for chemical components. As per this invention, the cohort whose feed is supplemented with D2-linoleic acid ethyl ester will show a significant reduction in the amount of 2-nonenal due to stabilization of the PUFAs against lipid chain auto-oxidation in dermal cells.

What is claimed is:

1. A composition suitable for topical application comprising an effective amount of one or more deuterated polyunsaturated fatty acids or esters thereof wherein the amount of said deuterated polyunsaturated fatty acid is sufficient to inhibit lipid auto-oxidation and thereby reduce skin damage.

2. The composition of claim 1, wherein said composition is a cosmetic composition.

3. The composition of claim 1, wherein said composition is a skin care or medicinal composition.

4. The composition of claim 1, wherein said deuterated polyunsaturated fatty acid or ester thereof is a deuterated linoleic acid or an ester thereof.

5. The composition of claim 1, wherein said deuterated polyunsaturated fatty acid or ester thereof is deuterated linolenic acid or an ester thereof.

6. The composition of claim 1, wherein said deuterated polyunsaturated fatty acid or ester thereof is a mixture of a deuterated linoleic acid or an ester thereof and a deuterated linolenic acid or an ester thereof.

7. The composition of claim 1, wherein said composition is a cream, an ointment, a lotion, a paste, an emoillent, a paste, a spray, or an emulsion.

8. The composition of claim 1, wherein the polyunsaturated fatty acid or ester thereof is present in an amount from about 0.1% w/w to about 25% w/w.

9. The composition of claim 1, further comprising one or more ingredients selected from the group consisting of a UV absorbing agent, a moisturizing agent, an antioxidant, a structuring agent, a thickener, an emulsifier, a silicone agent, a preservative, and a further active pharmaceutical ingredient/active agent.

10. A method for reducing dermal degradation of a patient's skin which method comprises applying to the skin a cosmetic or skin care composition comprising an effective amount of one or more deuterated polyunsaturated fatty acids or esters thereof in an amount effective to inhibit lipid auto-oxidation and subsequent damage to the dermis of the skin.

11. The method of claim 10, wherein said method attenuates the smell of aged skin.

12. The method of claim 10, wherein said composition is a cosmetic composition.

13. The method of claim 10, wherein said composition is a skin care composition.

14. The method of claim 10, wherein said deuterated polyunsaturated fatty acid or ester thereof is a deuterated linoleic acid or an ester thereof.

15. The method of claim 10, wherein said deuterated polyunsaturated fatty acid or ester thereof is deuterated linolenic acid or an ester thereof.

16. The method of claim 10, wherein said deuterated polyunsaturated fatty acid or ester thereof is a mixture of a deuterated linoleic acid or an ester thereof and a deuterated linolenic acid or an ester thereof.

17. The method of claim 10, wherein said composition is a cream, an ointment, a lotion, a paste, an emoillent, a spray, a paste, or an emulsion.
